(19) United States
(12) Patent Application Publication
(10) Pub. No.: US 2021/0361574 A1
(43) Pub. Date: Nov. 25, 2021

(54) NANOEMULSION CANNABIS FORMULATIONS AND METHODS OF MAKING SAME

(71) Applicant: NEW FRONTIER BREWING COMPANY, LLC, Anaheim, CA (US)

(72) Inventor: Michael SAWYER, Mission Viejo, CA (US)

(21) Appl. No.: 17/285,833

(22) PCT Filed: Oct. 15, 2019
(86) PCT No.: PCT/US2019/056301

§ 371 Date: Apr. 15, 2021

Related U.S. Application Data
(60) Provisional application No. 62/745,645, filed on Oct. 15, 2018.

(51) Int. Cl. A61K 9/107; A61K 47/26; A61K 47/44; A61K 47/40; A61K 36/185; A23L 33/105; A61K 8/06

(52) U.S. Cl. CPC A61K 9/1075; A61K 47/26; A61K 47/44; B82Y 5/00; A61K 36/185; A23L 33/105; A61K 8/068; A61K 47/40

(57) ABSTRACT

The invention provides a nanoemulsion formulation comprising an aqueous phase, an oil phase dispersed in the aqueous phase, and at least one surfactant. The oil phase includes a cannabis oil. The present invention also provides a preparation that comprises the nanoemulsion formulation. The preparation can be an edible, a beverage, or a pharmaceutical preparation. The present invention still further provides a method of preparing such formulations.

### RELATED APPLICATION

This application claims priority from U.S. Provisional Application No. 62/745,645, filed Oct. 15, 2018, the entire contents of which is incorporated herein by reference.

### TECHNICAL FIELD

The present invention relates to nanoemulsion formulations having a cannabis oil dispersed in water, as well as preparations comprising such formulations. The present invention further provides methods of making these formulations.

### SUMMARY

Delta-9-tetrahydrocannabinol (“THC”) is the principal psychoactive compound of cannabis. Medicinal and recreational cannabis products having THC as an active ingredient have been formulated in a variety of ways, including both edible products and oil-oil infusions. However, such conventional formulations have poor water solubility, and thus poor absorption, in the mucous membranes, gastrointestinal tract, and through the skin. This results in such products having low bioavailability and delayed onset of action. For example, it may take between 45 minutes and two hours before a user experiences effects from these types of conventional cannabis products. This can lead to overindulgence, for instance, where the user takes an additional dose based on a misguided belief that the initial dose was ineffective. In addition, such conventional formulations provide a long dose-response curve, with effects that can last longer than desired (e.g., more than 12 hours).

Still further, when a conventional product having THC is eaten or swallowed, the THC is subject to first-pass metabolism through the liver. First-pass metabolism is undesirable as it decreases bioavailability of the THC, and also converts the THC to 11-hydroxy-tetracannabinol (11-OH-THC). Whereas THC often provides a user with energetic and socially-compatible effects, 11-OH-THC may cause the user to feel sluggish, tired, and unable to complete normal tasks.

As set forth in the present disclosure, it would be desirable to provide a cannabis formulation having increased bioavailability and accelerated onset of effects. Additionally or alternatively, it would be desirable to provide a cannabis formulation that reduces or eliminates the isomerization of THC to 11-OH-THC by avoiding first-pass metabolism via the liver. It would also be desirable to provide a cannabis formulation that reduces the dose-response curve, thus allowing a user to return to baseline more quickly than with conventional cannabis products. In addition, it would be desirable to provide a cannabis formulation that has a particle size of less than 100 nm, has enhanced homogeneity and long term stability in suspension, provides ease of dispersion during commercial scale manufacturing, and that is also compatible with water.

A nanoemulsion formulation is disclosed herein. The nanoemulsion formulation comprises an aqueous phase, an oil phase dispersed in the aqueous phase, and at least one surfactant. The oil phase comprises a cannabis oil. The cannabis oil can comprise cannabinoids, terpenes, and combinations thereof. The oil droplets of the cannabis oil can have a mean particle diameter of less than 100 nanometers, or even less than 50 nanometers. The nanoemulsion formulation can be provided in either liquid or powder form.

This disclosure also provides a preparation that comprises the nanoemulsion formulation. The preparation can be an edible product or a topical product. For example, the preparation can be a food, a beverage, a pharmaceutical preparation, or a topically-applied cosmetic makeup preparation.

Still further, this disclosure provides a method of preparing the nanoemulsion formulation. The method comprises mixing water with a first surfactant to obtain an aqueous phase, and mixing a second surfactant, a cannabis oil, and a carrier oil to obtain an oil phase. The oil phase is heated and mixed until a homogenous mixture is formed. The aqueous phase is mixed and agitated until the aqueous phase reaches a minimum temperature, at which point the homogenous mixture is added to the aqueous phase to obtain a solution. The solution is agitated to obtain a reaction mixture, which can then be filtered to obtain the nanoemulsion formulation.

### DETAILED DESCRIPTION

The following detailed description is exemplary in nature and is not intended to limit the scope, applicability, or configuration of the invention in any way. Rather, the following description provides some practical illustrations for implementing exemplary embodiments of the present invention. Skilled artisans will recognize that the examples provided herein have many useful alternatives that fall within the scope of the invention.

Disclosed herein is a nanoemulsion formulation. An emulsion is a liquid-liquid dispersion where a first liquid acts as a continuous medium, and a second liquid acts as a dispersion medium of droplets in the first liquid. Emulsions with nanoscopic droplet sizes (typically in the range of 20-200 nm) are referred to as nanoemulsions.

An emulsion (including nanoemulsions) can be of two main types, including a water-in-oil emulsion or an oil-in-water emulsion. For a water-in-oil emulsion, oil is the continuous phase, and water is the dispersed phase (i.e., water is dispersed in oil). For an oil-in-water emulsion, water is the continuous phase, and oil is the dispersed phase (i.e., oil is dispersed in water).

The nanoemulsion formulation of the present disclosure comprises an aqueous phase, and an oil phase dispersed in the aqueous phase. Thus, the nanoemulsion formulation of the present disclosure is an oil-in-water emulsion. The oil phase refers to the internal hydrophobic core of the nanoemulsion where the oil is present, whereas the aqueous phase refers to an external phase of the nanoemulsion in which the oil phase is dispersed. The oil phase can refer to a single oil or to a mixture of oils.

The aqueous phase of the nanoemulsion formulation comprises water and can optionally comprise additional components. The aqueous phase is present in the nanoemulsion formulation in a weight ratio of between about 70% and about 95% (e.g., from about 75% to about 92%, or from about 80% to about 90%).

The oil phase of the nanoemulsion formulation comprises a cannabis oil. The cannabis oil can be present in the nanoemulsion formulation in a weight ratio of from about 0.1% to about 10% (e.g., from about 0.5% to about 8%, from about 0.75% to about 6%, from about 1% to about 4%, from about 1.5% to about 3%, or from about 1.8% to about 2.8%). In some cases, the cannabis oil comprises more than one cannabis oil. In embodiments of this nature, the recited weight ratios for the cannabis oil represent a total weight ratio for all cannabis oils present in the nanoemulsion formulation.

In some embodiments, the cannabis oil is a cannabinoid compound. The cannabinoid compound can be a single cannabinoid or a combination of two or more cannabinoids. Over 100 cannabinoids have been isolated from cannabis plants, including, e.g., THC, cannabidiol (CBD), cannabinol, tetrahydrocannabinolic acid, cannabidolic acid, cannabigerol, cannabichromeme, cannabicyclol, cannabivarin, tetrahydrocannabivarin, cannabidivarin, cannabichromevarin, cannabigerovarin, cannabigerol monomethyl ether, cannabielsoin, and cannabicitran. Any of these or other cannabinoids can be used as the cannabinoid in the nanoemulsion formulation. In some cases, the cannabinoid of the nanoemulsion formulation is THC, CBD, or a combination thereof. The one or more cannabinoids of the nanoemulsion formulation can be synthetically prepared or obtained naturally (e.g., extracted from a cannabis plant).

In some cases, the cannabis oil comprises one or more terpenes. Terpenes are volatile unsaturated hydrocarbons found in the essential oils of plants. Terpenes are not psychoactive, and can provide variable effects when provided in combination with one or more cannabinoids (particularly when combined with THC).

Any terpene can be used in the present nanoemulsion formulation, either alone or in combination with one or more other terpenes. Non-limiting examples of cannabis terpenes that can be used in the nanoemulsion formulation include myrcene, camphene, terpineol, valencene, geraniol, delta-3-carene, linalool, alpha-pinene, beta-pinene, alpha-bisabolol, limonene, eucalyptol, borneol, humulene, trans-nerolido, caryophyllene, and beta-caryophyllene. When the terpene is myrcene, the myrcene will advantageously increase cell permeability and allow cannabinoid compounds to be absorbed more quickly than without the myrcene. The terpenes of the nanoemulsion formulation can be synthetically prepared or obtained naturally.

The cannabis oil of the nanoemulsion formulation can be provided in various possible combinations. For instance, in some embodiments, the cannabis oil comprises one or more cannabinoids and no terpenes. In certain other embodiments, the cannabis oil comprises one or more terpenes and no cannabinoids. In still other embodiments, the cannabis oil comprises at least one cannabinoid and at least one terpene, and can comprise more than one cannabinoid and/or more than one terpene. In many embodiments, the cannabis oil comprises at least one cannabinoid comprising THC.

In some embodiments, the oil droplets of the cannabis oil have a particle diameter size of less than 100 nanometers. This particle diameter size of the cannabis oil can refer to the median particle diameter (D50), the z-average particle diameter, or the particle diameter of all oil droplets of the cannabis oil. In certain embodiments, the particle diameter size of the oil droplets of the cannabis oil is less than 50 nanometers (e.g., less than 45 nanometers, less than 40 nanometers, less than 35 nanometers, less than 30 nanometers, or even less than 25 nanometers). By controlling the droplet size of the nanoemulsion formulation to be within any of the above-noted ranges, fewer steps will be required for absorption of the cannabis oil than with conventionally-formulated cannabis products. This in turn causes quicker absorption of the cannabis oil. In addition, the small droplet size allows the nanoemulsion formulation to be kinetically stable in suspension for months to years, depending on the particular formulation. As used herein, kinetically stable means that the suspension will not separate or settle.

The nanoemulsion formulation further comprises at least one surfactant. The surfactant stabilizes the nanoemulsion by reducing interfacial tension between the oil phase and the water phase. In addition, the surfactant maintains the size of the droplets after they are formed. The surfactants can comprise any non-ionic surfactants, including, but not limited to, poloxamers, polysorbates, sodium lauryl sulfate, lauryl dimethyl amine oxide, cetyltrimethylammonium bromide, polyethoxylated alcohols, sucrose esters, octoxynol, N, N-dimethyldodecylamine-N-oxide, hexadecyltrimethylammonium bromide, polyoxyl 10 lauryl ether, bile salts (e.g., sodium deoxycholate and sodium cholate), polyoxyl castor oil, nonylphenol ethoxylate, cyclodextrins, lecithin, methylbenzethonium chloride, vitamin E tocopheryl polyethylene glycol 1000 succinate (vitamin E TPGS) and combinations thereof. When vitamin E TPGS is provided in the formulation, it serves not only as a surfactant, but also provides antioxidant properties to the formulation. Such properties help combat degradation of the formulation, and thereby promote its long-term storage and stability. Skilled artisans will be able to select suitable surfactants from any of these or other surfactants. In some cases, the surfactant is a polysorbate that comprises sorbitane monooleate (also known as Span 80) and/or polyoxyethylene (20) sorbitan monooleate (also known as Tween 80).

Often, the nanoemulsion formulation will comprise at least two surfactants, including a first surfactant that is present in the aqueous phase, and a second surfactant that is present in the oil phase. In some cases, where the nanoemulsion formulation includes both the first surfactant and the second surfactant, the first surfactant is Tween 80, and the second surfactant is Span 80. This, however, is not required, as alternative surfactants can be used without departing from the spirt and scope of the present invention.

The total weight ratio of all surfactants present in the nanoemulsion formulation is from about 0.01% to about 12%. For example, the total weight ratio of surfactants can be from about 2% to about 9%, from about 3% to about 8.5%, or from about 5% to about 8%.

The surfactants used in the nanoemulsion formulation can be selected, at least in part, based on the Hydrophile Lipophile Balance (HLB). The HLB is an empirical scale that provides an indication of the solubility of a surfactant. Each surfactant has an associated HLB value based on the HLB scale. The higher the HLB value, the more hydrophilic or water-soluble the surfactant is. The lower the HLB value, the more lipophilic or oil-soluble the surfactant is.

For a blend of surfactants (i.e., two or more surfactants in a single formulation), the HLB value of the blend is calculated based on a weighted average of HLB values for each surfactant. For example, if the formulation comprises 75.6% weight of Tween 80 (HLB value=15.0) and 24.4% of Span 80 (HLB value of 4.3), the HLB value of this blend of surfactants is approximately 12.9 [(0.244*4.3)+(0.756*15)].

The HLB value of surfactants in the present nanoemulsion formulation is specifically tailored to an HLB requirement for its particular application (e.g., oil-in-water emulsions). Such tailoring enables the least amount of surfactant to be used to achieve emulsification. In many embodiments, the HLB value of the surfactant (or blend of surfactants) of the present nanoemulsion formulation is from about 12 to about 18 (e.g., from about 12 to about 15; from about 12.5 to about 14; or from about 12.75 to about 13.5). In some cases, the HLB value of the surfactant (or blend of surfactants) in the nanoemulsion formulation is about 12.9.

In many embodiments, the oil phase comprises a carrier oil. The carrier oil can be selected to maximize gastrointestinal uptake of the formulation, and thus increase its bioavailability. Non-limiting examples of suitable carrier oils that can be used in the present formulation include olive oil, lemon oil, palm oil, grapeseed oil, sesame oil, canola oil, castor oil, peanut oil, corn oil, fish oil, coconut oil, mineral oil, vegetable oils, and combinations thereof.

In some embodiments, the oil droplets of the carrier oil have a particle diameter size of less than 100 nanometers. This particle diameter size of the carrier oil can refer to the median particle diameter (D50), the z-average particle diameter, or the particle diameter of all oil droplets of the carrier oil. In certain embodiments, the particle diameter size of the oil droplets of the carrier oil is less than 50 nanometers (e.g., less than 45 nanometers, less than 40 nanometers, less than 35 nanometers, less than 30 nanometers, or even less than 25 nanometers).

The carrier oil can be present in the nanoemulsion formulation in a weight ratio of between about 0.1% and about 12% (e.g., from about 1% to about 8%, from about 2% to about 6%, or from about 3.5% to about 5.5%). In certain embodiments, the carrier oil is present in the nanoemulsion formulation in a greater weight ratio than is the cannabis oil.

Although embodiments comprising a carrier oil have been described herein, in certain embodiments, the formulation does not comprise both a surfactant and a carrier oil. For example, in embodiments where vitamin E TPGS is the at least one surfactant, the formulation may not include a separate carrier oil. In such embodiments, the at least one surfactant (e.g., the vitamin E TPGS) can serve as the carrier oil of the formulation. In embodiments of this nature, the weight ratio of the at least one surfactant present in the formulation is increased (e.g., relative to a formulation having a separate carrier oil) to account for the lack of a separate carrier oil.

The weight ratio of total surfactants to total oil in the nanoemulsion formulation can be from about 2.5:1 to about 1:2.5. As used herein, total oil refers to a total amount of both the cannabis oil and the carrier oil in the nanoemulsion formulation. In some embodiments, the surfactant-to-oil weight ratio is about 2:1, about 1.5:1, about 1:1, about 1:1.5, or about 1:2.

The nanoemulsion formulation of the present disclosure provides many advantages over conventional cannabis products. For instance, the present nanoemulsion formulation is able to reduce or avoid first pass metabolism, and thus provide enhanced bioavailability. This in turn provides a quicker onset of action (e.g., about 100 times faster) than conventional cannabis formulations. In particular, this quicker onset of action allows a user to feel effects from the active ingredients within minutes (such as within 3 minutes, within 2 minutes, or within 1 minute), or even within 20-30 seconds of ingesting the nanoemulsion formulation or applying it to the user's skin. This is in contrast to conventional edible formulations, where the user must wait for the product to be fully digested for the user to feel its effects.

In addition, the present nanoemulsion formulation provides a dose-response curve that is similar to that of alcohol, allowing a user to return to baseline within 30-90 minutes after ingesting the nanoemulsion formulation or applying it to the user's skin. The time to return to baseline will depend on both the particular dosage and the user's metabolism.

Still further, as discussed in greater detail below, the nanoemulsion formulation enables users to feel desired psychoactive effects using a lower dose of active ingredients than with conventional cannabis formulations. In some cases, the nanoemulsion formulation comprises active ingredients in an amount that is about 10-20 times less than that used for conventional cannabis formulations.

In some embodiments, the present disclosure provides a preparation of the nanoemulsion formulation. The preparation can be a solid, liquid, or gel product that comprises (e.g., incorporates) the nanoemulsion formulation. The preparation can be a water-based product. In this manner, the nanoemulsion preparation provides an advantage over oil-oil infusions, since oil-oil infusions are incompatible with (e.g., insoluble in) water.

In certain embodiments, the preparation is a topical product. Such topical products can include, but are not limited to, lotions, gels, soaps, shampoos, body sprays, creams, ointments, face masks, sexual lubricants, and transdermal patches. Topical products of the present disclosure can also comprise any topically-applied cosmetic makeup product configured to provide any level of coverage, such as foundation, setting spray, blush, highlighter, lipstick, concealer, transdermal primer, and BB cream (also known as blemish balm cream or beauty balm cream). Such topical products can comprise liquid and solid products, including powder and/or mineral-based products.

In other embodiments, the preparation is an ingestible product. Such ingestible products can comprise, for example, beverages, edibles, and pharmaceutical preparations. Non-limiting examples of such preparations include non-alcoholic beer and wine, soda, juice, nutritional wellness “shot” drinks, water, sparkling water, energy drinks, coffee, tea, milk (dairy and non-dairy milk products, including almond milk and soy milk), hard and soft candies, chocolate, baked goods, confections, capsules, tablets, powders, sublingual tinctures, intranasal sprays, intravenous liquids, and intranasal powders.

The preparation can be provided in a dosage form in which the cannabis oil is present in the preparation in an amount from about 100 micrograms to about 50 milligrams. For example, in some cases, the cannabis oil is present in the preparation in an amount from about 200 micrograms to about 25 milligrams, or from about 300 micrograms to about 20 milligrams. In certain other embodiments, the cannabis oil is present in the preparation in an amount from about 500 micrograms to about 10 milligrams. In some embodiments, these ranges of cannabis oil refer to the amount of THC present in the preparation. However, it should be understood that these ranges can refer to the amount of any cannabis oil present in the preparation and are not limited to THC.

In some embodiments, the THC is present in the preparation in an amount less than 1 mg. For example, the THC can be present in the preparation in an amount less than 1 mg and greater than or equal to about 500 micrograms, such as from about 600 micrograms to about 900 micrograms, or from about 650 micrograms to about 850 micrograms.

In other embodiments, the THC is present in the preparation in an amount greater than 1 mg. For example, the THC can be present in the preparation in an amount greater than 1 mg and up to about 10 mg. For example, the THC can be present in the formulation in an amount from about 2 mg to about 9 mg, or from about 3 mg to about 8 mg.

The amount of the nanoemulsion formulation to be used in the preparation will depend on the level of effects desired. For instance, providing the present nanoemulsion formulation with a dosage of THC at about 500 micrograms provides minimal psychoactive effects, whereas providing the present nanoemulsion formulation with a dosage of THC at about 10 mg provides very strong psychoactive effects. In contrast, conventional oil-oil infusions often require active ingredients (e.g., THC) in dosage amounts of from 5 mg to 100 mg (or even more than 500 mg) for users to feel desired psychoactive effects. Thus, in this manner, the dose of active ingredients used in the present preparation can be significantly less than the dose of active ingredients used in conventional cannabis products comprising oil-oil infusions.

In addition, where the preparation comprises both THC and terpenes, the present nanoemulsion formulation ensures that the preparation includes the specific terpene profile of the cannabis oil being used in the formulation, and further ensures that the user experiences the effects of this specific terpene profile. As used herein, the phrase “terpene profile” refers to the combination of specific types and amounts of both THC and terpenes that are present in the cannabis oil. This feature of the present disclosure advantageously allows strain-specific food and beverages to be prepared. This is in contrast to conventionally-formulated edibles, where THC is isomerized to 11-OH-THC during absorption of the edible product. Accordingly, with such conventional products, the terpene profile changes before a user experiences any of its effects. This issue with conventionally-formulated cannabis products is addressed by the present nanoemulsion formulation and its related preparations.

The present invention further provides a method of preparing the nanoemulsion formulation described above. The nanoemulsion formulation can be provided in either liquid or powder form. Methods of preparing both liquid and powder forms of the nanoemulsion formulation are described below.

### Preparation of the Aqueous Phase

Distilled water is added to a suitable mixing vessel. A pump can be used to circulate the water, and sonication (e.g., ultra-sonication) can be used to agitate the water particles. As used herein, ultra-sonication refers to the use of frequencies at about 20 kHz and greater.

The first surfactant is then added to the mixing vessel to obtain the aqueous phase. In certain embodiments, the first surfactant is Tween 80. This, however, is by no means required. Examples of other suitable surfactants that can be used as the first surfactant are discussed above.

### Preparation of the Oil Phase

The method further comprises mixing together a second surfactant, a cannabis oil, and a carrier oil to obtain an oil phase. This mixing is performed in a container that is separate from the mixing vessel such that the aqueous phase and the oil phase are prepared separately. In some embodiments, the second surfactant is Span 80; the carrier oil is olive oil; and the cannabis oil is THC, CBD, terpenes, or a combination thereof. Other examples of suitable surfactants, cannabis oils, and carrier oils that can alternatively be used in the nanoemulsion formulation are discussed above.

The method further comprises heating and mixing the oil phase until a homogenous mixture is formed. Homogeneous is used herein in accordance with its ordinary and plain meaning to refer to a mixture that is completely uniform throughout and has no separation of its components. Thus, the homogeneous mixture of the present disclosure has the same proportion of components in any given sample.

### Preparation of Liquid Nanoemulsion Formulation

In a further step, the method comprises mixing and agitating (e.g., sonicating) the aqueous phase until the aqueous phase reaches a minimum temperature, such as about 52.5 degrees Celsius. Once the aqueous phase reaches the minimum temperature, the homogenous suspension is added to the aqueous phase to obtain a solution. The solution is then agitated (e.g., sonicated) for at least 60 minutes in order to obtain a reaction mixture. During this time period, the temperature is modulated such that the temperature of the solution cycles between a pre-specified temperature range. In some embodiments, this temperature range is between about 60 degrees Celsius and about 52 degrees Celsius.

During the sonication process, a small sample of the solution is collected using a pipette or any other suitable instrument. The particle size of the sample (i.e., size of oil droplets in the sample) is checked using a particle size analyzer to ensure that a minimum particle size is reached. Any known particle size analyzer can be used, including a laser diffraction particle size analyzer. In some embodiments, sonication continues until the mean diameter (i.e., D50) of the cannabis oil is less than 100 nanometers. In other embodiments, sonication continues until the mean diameter of the cannabis oil is less than 50 nanometers. The reaction mixture that has the desired minimum particle size is thereafter filtered to remove contaminants created during the sonication process, thereby obtaining a filtered nanoemulsion in liquid form.

### Preparation of Powdered Nanoemulsion Formulation

To convert the liquid nanoemulsion into a powdered nanoemulsion form, the filtered, liquid nanoemulsion can be mixed with a solid hydrophilic excipient (e.g., a sugar). In some embodiments, the filtered nanoemulsion is mixed with a cyclodextrin powder or a maltodextrin powder in order to obtain a homogenous slurry. In some cases, the filtered nanoemulsion is mixed with the sugar (e.g., the cyclodextrin or maltodextrin powder) in a weight ratio of between about 2:1 to about 1:1, such as an about 2:1 weight ratio, an about 1.75:1 weight ratio, an about 1.5:1 weight ratio, an about 1.25:1 weight ratio, or an about 1:1 weight ratio. The homogenous slurry can then be dried in any conventional manner (e.g., spray drying or freeze drying) in order to produce a dry powder.

### Example 1

### Preparation of Liquid Extract—Formulation A

Distilled water is added to a mixing vessel, and a pump is turned on to circulate the water. Sonication of the mixing vessel is started and occurs at an electrical current of 20 kHz or greater. Tween 80 (the water phase surfactant) is then added to the mixing vessel.

The oil phase is prepared in a separate container by combining olive oil, a cannabis extract, and Span 80 (the oil phase surfactant). The cannabis extract is a whole plant, full-spectrum extract that comprises CBD, THC, other cannabinoids, and terpenes. The oil phase is heated and stirred until a completely homogenous suspension is formed having no separation. Once the oil phase is fully prepared, the water phase is allowed to reach a temperature of approximately 52.5 degrees Celsius while being mixed and sonicated. The oil phase is then added into the mixing vessel containing the water phase.

Once both the water and oil phases are combined, the sonication continues for at least 60 minutes. During this process, the temperature of the solution must be modulated so as to reach a target upper temperature, held for a length of time, and then allowed to cycle down to a lower temperature. The solution is held at the target upper temperature for about a minute before the temperature reaches the lower temperature. This temperature cycling is repeated throughout the entire sonication process. The target upper temperature can be a range (e.g., between approximately 55 degrees Celsius and approximately 60 degrees Celsius). Likewise, the lower temperature can also be a range (e.g., between approximately 52 degrees Celsius and approximately 55 degrees Celsius).

During the sonication process, a small sample is collected with a pipette. The particle size of this sample (i.e., size of oil droplets in the sample) is checked using a laser diffraction particle size analyzer. Once a desired or minimum particle size is reached, the solution is filtered to remove any contamination created during the sonication process. This is accomplished using a sterile vacuum filtration flask. The finished filtered sterilized solution is put into sterile containers and refrigerated until ready to use.

The amounts of Tween 80, Span 80, Extract, Olive Oil, and Water used in these above-described steps to prepare Formulation A are shown in Table 1 below.

TABLE 1
Formulation A

| Tween | | Span | | | | Olive | | | | Total |
|---|---|---|---|---|---|---|---|---|---|---|
| 80 | | 80 | | Extract | | Oil | | Water | | Mass |
| [g] | % | [g] | % | [g] | % | [g] | % | [g] | % | [g] |
| 157.1 | 5.8 | 37.9 | 1.4 | 65.0 | 2.4 | 130.1 | 4.8 | 2318.0 | 85.6 | 2708.1 |

### Example 2

### Preparation of Liquid Isolate—Formulation B

Example 1 is repeated, except Formulation B of Example 2 (shown in Table 2 below) is used instead of Formulation A of Example 1.

TABLE 2
Formulation B

| Tween | | Span | | | | Olive | | | | Total |
|---|---|---|---|---|---|---|---|---|---|---|
| 80 | | 80 | | Isolate | | Oil | | Water | | Mass |
| [g] | % | [g] | % | [g] | % | [g] | % | [g] | % | [g] |
| 241.6 | 5.8 | 57.7 | 1.4 | 99.7 | 2.4 | 199.5 | 4.8 | 3558.0 | 85.6 | 4156.5 |

As used in this Example, the Isolate refers to an isolated cannabis compound having purity levels of 95% and higher.

### Example 3

### Preparation of Powdered Extract—Formulation C

Distilled water is added to a mixing vessel, and a pump is turned on to circulate the water. Sonication of the mixing vessel is started and occurs at an electrical current of 20 kHz or greater. The water-phase surfactant (Tween 80) is then added to the mixing vessel.

The oil phase is prepared in a separate container by combining olive oil, a cannabis extract, and Span 80 (the oil phase surfactant). The cannabis extract is a whole plant, full-spectrum extract that comprises CBD, THC, other cannabinoids, and terpenes. The oil phase is heated and stirred until a completely homogenous suspension is formed having no separation. Once the oil phase is fully prepared, the water phase is allowed to reach a temperature of approximately 52.5 degrees Celsius while being mixed and sonicated. The oil phase is then added into the mixing vessel containing the water phase.

Once both the water and oil phases are combined, the sonication continues for at least 60 minutes. During this process, the temperature of the solution must be modulated so as to reach a target upper temperature, held for a length of time, and then allowed to cycle down to a lower temperature. The solution is held at the target upper temperature for about a minute before the temperature reaches the lower temperature. This temperature cycling is repeated throughout the entire sonication process. The target upper temperature can be a range (e.g., between approximately 55 degrees Celsius and approximately 60 degrees Celsius). Likewise, the lower temperature can also be a range (e.g., between approximately 52 degrees Celsius and approximately 55 degrees Celsius).

During the sonication process, a small sample is collected with a pipette. The particle size of this sample (i.e., size of oil droplets in the sample) is checked using a laser diffraction particle size analyzer. Once the desired or minimum particle size is reached, the solution is filtered to remove any contamination created during the sonication process. This is accomplished using a sterile vacuum filtration flask.

The finished filtered nanoemulsion is mixed at a 1:1 weight ratio with cyclodextrin or maltodextrin powder to create a homogenous slurry. The slurry is fed into a spray dryer or a freeze dryer (lyophilizer) to remove all water present in the slurry so as to produce a fine dry powder. The finished powder is then stored under vacuum or in a 0% humidity environment until ready for use.

The amounts of Tween 80, Span 80, Extract, Olive Oil, and Water used in these above-described steps to prepare Formulation C are shown in Table 3 below.

TABLE 3
Formulation C

| Tween | | Span | | | | Olive | | | | Total |
|---|---|---|---|---|---|---|---|---|---|---|
| 80 | | 80 | | Extract | | Oil | | Water | | Mass |
| [g] | % | [g] | % | [g] | % | [g] | % | [g] | % | [g] |
| 157.1 | 5.8 | 37.9 | 1.4 | 65.0 | 2.4 | 130.1 | 4.8 | 2318.0 | 85.6 | 2708.1 |

### Example 4

### Preparation of Powdered Isolate—Formulation D

Example 3 is repeated, except Formulation D (shown in Table 4 below) is used instead of Formulation C of Example 3.

TABLE 4
Formulation D

| Tween | | Span | | | | Olive | | | | Total |
|---|---|---|---|---|---|---|---|---|---|---|
| 80 | | 80 | | Isolate | | Oil | | Water | | Mass |
| [g] | % | [g] | % | [g] | % | [g] | % | [g] | % | [g] |
| 241.6 | 5.8 | 57.7 | 1.4 | 99.7 | 2.4 | 199.5 | 4.8 | 3558.0 | 85.6 | 4156.5 |

As used in this Example, the Isolate refers to an isolated cannabis compound having purity levels of 95% and higher.

Various examples have been described with reference to certain disclosed embodiments. The embodiments are presented for purposes of illustration and not limitation. One skilled in the art will appreciate that various changes, adaptations, and modifications can be made without departing from the scope of the invention.

1. A nanoemulsion formulation comprising:
an aqueous phase;
an oil phase dispersed in the aqueous phase, the oil phase comprising a cannabis oil; and
at least one surfactant.

2. The nanoemulsion formulation of claim 1 wherein oil droplets of the cannabis oil have a mean particle diameter of less than 100 nanometers.

3. The nanoemulsion formulation of claim 2 wherein the mean particle diameter of the oil droplets of the cannabis oil is less than 50 nanometers.

4. The nanoemulsion formulation of claim 1 wherein the cannabis oil is selected from cannabinoids, terpenes, and combinations thereof.

5. The nanoemulsion formulation of claim 1 wherein the cannabis oil is selected from cannabidiol, 9-delta-tetrahydrocannabinol, and combinations thereof.

6. The nanoemulsion formulation of claim 1 wherein the at least one surfactant comprises a first surfactant that is present in the aqueous phase and a second surfactant that is present in the oil phase.

7. The nanoemulsion formulation of claim 1 wherein the at least one surfactant is selected from sorbitane monooleate, polyoxyethylene (20) sorbitan monooleate, and combinations thereof.

8. The nanoemulsion formulation of claim 1 wherein the oil phase comprises a carrier oil.

9. The nanoemulsion formulation of claim 8 wherein the carrier oil is olive oil.

10. The nanoemulsion formulation of claim 1 wherein the nanoemulsion formulation is in liquid form.

11. The nanoemulsion formulation of claim 1 wherein the nanoemulsion formulation is in powder form.

12. A preparation of the nanoemulsion formulation of claim 1 wherein the preparation comprises the nanoemulsion formulation, and the preparation is an edible, a beverage, a pharmaceutical preparation, or a cosmetic makeup product.

13. The preparation of claim 12 wherein the beverage is beer or wine.

14. The preparation of claim 12 wherein the pharmaceutical preparation is a tablet or capsule.

15. The preparation of claim 12 wherein the cannabis oil comprises 9-delta-tetrahydrocannabinol, and the 9-delta-tetrahydrocannabinol is present in the preparation in an amount of from about 500 micrograms to about 10 milligrams.

16. A method of preparing a nanoemulsion formulation, the method comprising:
mixing water with a first surfactant to obtain an aqueous phase;
mixing a second surfactant, a cannabis oil, and a carrier oil to obtain an oil phase;
heating and mixing the oil phase until a homogenous mixture is formed;
mixing and agitating the aqueous phase until the aqueous phase reaches a minimum temperature;
after the aqueous phase reaches the minimum temperature, adding the homogenous mixture to the aqueous phase to obtain a solution;
agitating the solution to obtain a reaction mixture; and
filtering the reaction mixture to obtain a filtered nanoemulsion formulation.

17. The method of claim 16 further comprising mixing the filtered nanoemulsion formulation with a cyclodextrin powder or a maltodextrin powder to obtain a homogenous slurry.

18. The method of claim 17 wherein the filtered nanoemulsion formulation is mixed with the cyclodextrin powder or the maltodextrin powder in about 1:1 weight ratio.

19. The method of claim 17 further comprising drying the homogenous slurry to produce a dry powder.

20. The method of claim 16 wherein the cannabis oil is selected from terpenes, cannabidiol, delta-9-tetrahydrocannabinol, and combinations thereof.

21. The method of claim 16 wherein the carrier oil comprises olive oil.

22. (canceled)

23. The nanoemulsion formulation of claim 1 wherein the at least one surfactant comprises vitamin E TPGS.

24. The nanoemulsion formulation of claim 1 wherein the formulation is a topical formulation configured to provide an onset of action within two minutes of applying the formulation to a user's skin.

25. The nanoemulsion formulation of claim 1 wherein the formulation is an oral formulation configured to provide an onset of action within two minutes of when a user orally ingests the formulation.
